(19) United States
(12) Patent Application Publication
(10) Pub. No.: US 2022/0217991 A1
(43) Pub. Date: Jul. 14, 2022

(54) SYSTEM AND METHOD OF MANUFACTURING SHELF-STABLE GELATO

(71) Applicant: Roses Confections, LP, New York, NY (US)

(72) Inventors: Brent Meyer, Dallas, TX (US); Yaacov Afek, Englewood, NJ (US)

(73) Assignee: Roses Confections, LP, New York, NY (US)

(21) Appl. No.: 17/146,559

(22) Filed: Jan. 12, 2021

(51) Int. Cl. A23G 9/08; A23G 9/40; A23G 9/34; A23L 3/44; A23L 3/3418; B65B 25/00; G01N 33/04

(52) U.S. Cl. CPC A23G 9/083; A23G 9/40; A23G 9/34; A23V 2002/00; A23L 3/3418; B65B 25/007; G01N 33/04; A23L 3/44

(57) ABSTRACT

A method of manufacturing shelf-stable gelato is provided. The method may include blending cream and sugar in a proportion. The blend may be frozen into units. The units may be freeze-dried in order to remove water content.

### BACKGROUND

Conventional gelato is a popular milk and sugar treat which is similar to ice cream, but with a greater density and richness, and it tends to be served at a slightly higher temperature. The history of making gelato is long, and many changes have been incorporated into the process, but today, conventional gelato is made using the so-called “Hot Process”.

The Hot Process involves first pasteurizing the ingredients by heating them up to 85 degrees Celsius, typically for five seconds, and then deceasing the temperature to 5 degrees Celsius. This pasteurizing step is necessary to increase the shelf-life of the resulting gelato. After pasteurization, the ingredients are then frozen while simultaneously being stirred via a machine called a “batch freezer”. The purpose of the stirring is to permit air molecules to enter the gelato, the water vapor of which is then frozen into ice crystals. These ice crystals are responsible for the smooth, silky texture of conventional gelato, and it is expected that gelato will be smoother and silkier than ice cream.

Another method of making conventional gelato is the so-called “Cold Process”. Instead of pasteurizing the ingredients, radiated or otherwise already sterilized ingredients are used. Otherwise, the Cold Process is similar to the Hot Process in that it also uses a batch freezer.

The third method of making conventional gelato is the so-called “Sprint Process”, in which the ingredients are all pre-packaged, except for water, which is added just before all of the ingredients are entered into the batch freezer.

These three methods are substantially similar and produce a nearly identical gelato. The lack of variation in the methods of producing gelato is responsible for the lack of variation in gelato generally, which is compounded by its similarity to ice cream, with which it is frequently confused. Conventional gelato may be creamier than ice cream, but ice cream is itself quite creamy. Conventional gelato may be served at a slightly higher temperature than ice cream, but it will nonetheless similarly melt at room temperature, and requires refrigeration. Unfortunately, freezing will not preserve the texture and flavor, because the chemical structure of gelato is very sensitive to changes in temperature.

The methods of making conventional gelato are very effective at producing a fresh food product. However, they are not effective at producing a product that will remain in its prime condition. The pasteurization step of the Hot Process and the pre-sterilized ingredients of the Cold Process do contribute to shelf life, but they do not prevent melting, and the water content provides a perfect environment for microbial life.

### SUMMARY

In accordance with the present invention, the disadvantages and limitations of the prior art are substantially avoided by providing a system and method for manufacturing shelf-stable gelato (“the manufactured gelato”). The manufactured gelato may be stored at room temperature in a sealed container, and therefore does not need to be refrigerated. The container may be irradiated or otherwise sterilized before being vacuum-sealed in order to prevent bacterial growth therein. The gelato manufactured will not melt at room temperature because of the changes made to the chemical or structural shape of the processed ingredients. Unlike conventional gelato, which is creamy, smooth or silky, dense, and elastic, the manufactured gelato is light, airy, crispy, and crunchy.

A first ingredient of the method may include a dairy product commonly referred to as half & half. There are many food items referred to generically as “half and half”, but the food item referred to herein is a cream typically used in coffee and made of equal parts whole milk and heavy cream. Cream is a dairy product composed of the higher-fat layer skimmed from the top of milk before homogenization, and therefore half and half is entirely dairy. Although the substance derived from skimming whey is also called cream, this type of cream is not the cream typically found in what is conventionally called half and half. Half and half is a convenience item, sold by many food producing companies, and is commonly found in supermarkets, grocers, and other food stores.

Milk is a nutrient-rich liquid food produced by the mammary glands of mammals, particularly cows and goats. Whole milk is 3.5% butterfat, whereas 2 percent milk is a low-fat variation with 2% butterfat. One percent milk and skim milk are even more low-fat variations, with 1% and 0.5% butterfat, respectively. Milk and heavy cream are similar except that they have different concentrations of butterfat, and are better understood as representing points on a water-butterfat spectrum, with skim milk on one end and butter on the other. As such, any milk and cream can be combined to form another milk or cream of a butterfat quantity between them on the water-butterfat spectrum.

Half and half is considered a light cream. As a light cream, half and half is around 20% butterfat, as opposed to whipping cream, which is around 35% butterfat, and heavy cream, which is around 38% butterfat. The concentration of butterfat is responsible for the different properties of light cream, whipping cream, and heavy cream. For example, while whipping cream and heavy cream can both be whipped to a peak, light cream cannot because whipping expands the volume of cream, and sufficient butterfat content is necessary to permit the increased volume to be sustained.

As discussed above, half and half may be made with one part whole milk and one part heavy cream, and it is also possible to substitute different kinds of milk and cream to produce a cream identical to half and half in butterfat content, even though the proportions will not be the same as whole milk and heavy cream—i.e., the substitute milk and cream will not be equal to each other in proportion. For example, it is possible to combine ⅔ cup of low-fat milk and ⅓ cup of heavy cream to obtain the desired 20% butterfat content of half and half. It is also possible to substitute cream for butter, with the proper proportion for half and half being 1 cup of whole milk and 1 tablespoon of butter.

A second ingredient of the method may include sugar, which is essentially a sweet tasting carbohydrate that is already found in products on the water-butterfat spectrum described above. While sugar commonly occurs naturally in unprocessed food items, additional sugar is often added to increase not only the sweetness or flavor but also to improve the “mouthfeel”, which is the physical sensations, separate from the flavor itself, that one experiences when food is in the mouth. The improvement of the mouthfeel is at least in part because sugar acts as a bulking agent, (i.e., it adds bulk to food), thereby increasing the texture quality. This bulking characteristic also assists in maintaining the volume of a food item.

Additionally, sugar acts as a preservative against food decay and prolongs the shelf life of food products. The preservative quality of sugar works because of its humectant character, which maintains and stabilizes the water content in a food. By stabilizing the water content, sugar slows down the growth of various microbial life forms, such as bacteria, mold, and yeast.

Other characteristics of sugar that may be utilized in the present system and method include its viscosity adding attribute, which is based on its character as a bulking agent, and its anticoagulant attribute, which delays the coagulation of proteins. Coagulation occurs when cream or milk is boiled. One way in which the present system and method prevent coagulation is by preventing boiling, and another is the inclusion of sugar.

Sugar is a broad category of sweet carbohydrates that includes simple sugars or monosaccharides, such as glucose, fructose, and galactose, and so-called “double sugars” or disaccharides, such as sucrose, lactose, and maltose. Double sugars are compounds of two monosaccharides, with sucrose formed from glucose and fructose, lactose formed from glucose and galactose, and maltose formed from two molecules of glucose. There are also saccharide compounds featuring more than two monosaccharides, which are therefore called polysaccharides; however, polysaccharides are generally not referred to as sugars. Sucrose, which is the primary sugar in so-called “table sugar”, is generally extracted from certain plants, such as beets and sugarcane, in the form of raw sugar crystals, whereafter it is refined by washing the sugar crystals and dissolving them into a syrup. This syrup is filtered to improve its purity. The syrup is then desaturated of color by being passed over carbon. The syrup is boiled to increase its concentration and reduce it to crystals. These odorless, colorless, and sweet crystals constitute the table sugar used in the system and method herein, and will be referred to hereafter as simply “sugar”.

Half and half is generally about 4% lactose, which as described previously, is also a sugar. However, the saturation point, which is the concentration level at which the solubility (of half and half) is at equilibrium, is significantly above 4%, and therefore additional sugar can be dissolved.

Half and half may be combined with additional sugar via a blending process. If sugar is merely poured into half and half at room temperature, it will simply settle on the bottom of the receiving vessel, possibly occasioning some passive dissolving. To speed the blending process, energy should be adding into mixture. Energy may be generated in the form of turbulence and/or heat. Thus, the sugar may be added to the half and half in a blender, food processor, or mixing bowl and then the half and half and the sugar may be actively and mechanically blended together. It is important that the heat is applied slowly, to prevent boiling or a Maillard reaction, which is a chemical reaction when protein reacts with sugars, effectively changing the butterfat's flavor and color. Alternatively, the sugar may be mixed with the half and half by using a manually operated utensil, such as a spoon, spatula, or whisk. In addition to mixing and as a prior, concurrent, or subsequent step, the ingredients may be heated in order to facilitate the blending process. The volumetric proportion of half and half to the added sugar is about 9 to 1, respectively. The blending should be sufficiently rigorous to generate enough heat to dissolve the sugar and, if necessary, pasteurize the half & half.

The dissolved blend is then frozen into cubes. Frozen cubes can be formed by pouring liquid in a plastic tray and placing the tray in a freezer, but this is an inefficient process that exposes the frozen cubes to freezer burn and requires the cubes to be forcibly removed from the tray. Another method is to use an icemaker, which separates the cubes from the cube mold using (a small amount of) heat and physically ejects the cubes using ejector blades. The cubes may be 1″ cubed.

The rate at which the blend freezes is important because it affects the size of the (water) crystals. A slower freeze rate results in larger crystals, which are more easily sublimated during the freeze dry process, and a slower freeze rate results in smaller crystals, which can be sublimated without damaging the product texture. The freeze rate, temperature profile, and duration may be conventional for standard ice cream making.

While the blend is freezing, flavors and additional ingredients, such as pistachio, lemon, chocolate, strawberry, etc., may be slowly added so as to ensure that they do not simply sink to the bottom of any given tray.

After the cubes are frozen, they may then be freeze dried. Freeze drying is a process by which material, such as food, is dehydrated at low temperatures. It may include first freezing the food by placing the cubes in a deep freezer, commonly referred to as an IQF (Individual Quick Freeze), lowering the pressure, then removing the (frozen) water content by converting it from a solid to a gas.

Typically, dehydration occurs by increasing the temperature sufficiently to cause evaporation—more precisely, vaporization of liquids from the surface or boiling from the interior. If it is desirable to dehydrate water from a product containing ice, the ice is first melted via heat, and then heated further to bring about the vaporization and boiling described. Unfortunately, the application of heat may have undesirable effects on the structure and composition of the product. However, since the state or phase of matter depends on the pressure as well as the temperature, water may be transitioned from solid to gas with only a minimal use of heat in a process called sublimation.

Sublimation may be effected by reducing the pressure below the substance's “triple point”, which is the point at which the three phases of the substances occur in an equilibrium because the sublimation curve, fusion curve, and vaporization curve intersect. The pressure may be set to the standard in freeze drying. This intersection is important because it provides a pathway by which water can transition between the solid state of ice and the gas state of water vapor without passing through the liquid state, which may damage the product due to the volatility of boiling water, particularly as the boiling water flows through the product medium. As the water in the butterfat-rich product boils, the butterfats may undergo the Maillard reaction, which may have undesirable effects on the manufactured gelato.

Only a small amount of heat is necessary for sublimation, and this heat should be applied slowly in order to damage the product's structure. The temperature and application of heat may be conventional according to standard freeze drying. A slow application of heat allows the energy to be distributed throughout rather than narrowly, intensely, and continuously on any given location. Since the heat will be applied in a low-pressure environment, the heat will mainly occur via conduction or radiation instead of convection.

By reducing the pressure sufficiently, the water molecules have enough room to move away from each other. The minimal amount of heat needed allows the water molecules to overcome their mutual attractive forces, separate, and enter the gas phase. The water vapor, as gas, can be collected and finally separated from the now dehydrated product.

The finished gelato product may be treated with a nitrogen flush and packaged in zip-top laminated stand-up pouches, commonly referred to as flexible packaging. Dessicant powder packs may be included in the packaging to absorb any excess moisture.

### BRIEF DESCRIPTION OF THE DRAWINGS

FIG. 1 shows an exemplary method of acquiring and preparing the dairy components.

FIG. 2 shows an exemplary method of acquiring and preparing the sucrose component.

FIG. 3 shows an exemplary method of blending the sucrose with the dairy components.

FIG. 4 shows an exemplary method of freezing and freeze-drying the blend.

FIG. 5 shows an exemplary method of adding flavor.

FIG. 6 shows an exemplary method of adding flavor.

FIG. 7 shows an exemplary method of controlling for crispiness.

FIG. 8 shows an exemplary method of regulating the temperature-pressure profile of the freeze drier.

FIG. 9 shows an exemplary method of packaging the product.

### DETAILED DESCRIPTION

A first ingredient of the method may include a dairy product commonly referred to as half & half. There are many food items referred to generically as “half and half”, but the food item referred to herein is a cream typically used in coffee and made of equal parts whole milk and heavy cream. Cream is a dairy product composed of the higher-fat layer skimmed from the top of milk before homogenization, and therefore half and half is entirely dairy. Although the substance derived from skimming whey is also called cream, this type of cream is not the cream typically found in what is conventionally called half and half. Half and half is a convenience item, sold by many food producing companies, and is commonly found in supermarkets, grocers, and other food stores.

Milk is a nutrient-rich liquid food produced by the mammary glands of mammals, particularly cows and goats. Whole milk is 3.5% butterfat, whereas 2 percent milk is a low-fat variation with 2% butterfat. One percent milk and skim milk are even more low-fat variations, with 1% and 0.5% butterfat, respectively. Milk and heavy cream are similar except that they have different concentrations of butterfat, and are better understood as representing points on a water-butterfat spectrum, with skim milk on one end and butter on the other. As such, any milk and cream can be combined to form another milk or cream of a butterfat quantity between them on the water-butterfat spectrum.

Half and half is considered a light cream. As a light cream, half and half is around 20% butterfat, as opposed to whipping cream, which is around 35% butterfat, and heavy cream, which is around 38% butterfat. The concentration of butterfat is responsible for the different properties of light cream, whipping cream, and heavy cream. For example, while whipping cream and heavy cream can both be whipped to a peak, light cream cannot because whipping expands the volume of cream, and sufficient butterfat content is necessary to permit the increased volume to be sustained.

As discussed above, half and half may be made with one part whole milk and one part heavy cream, and it is also possible to substitute different kinds of milk and cream to produce a cream identical to half and half in butterfat content, even though the proportions will not be the same as whole milk and heavy cream—i.e., the substitute milk and cream will not be equal to each other in proportion. For example, it is possible to combine ⅔ cup of low-fat milk and ⅓ cup of heavy cream to obtain the desired 20% butterfat content of half and half. It is also possible to substitute cream for butter, with the proper proportion for half and half being 1 cup of whole milk and 1 tablespoon of butter.

A second ingredient of the method may include sugar, which is essentially a sweet tasting carbohydrate that is already found in products on the water-butterfat spectrum described above. While sugar commonly occurs naturally in unprocessed food items, additional sugar is often added to increase not only the sweetness or flavor but also to improve the “mouthfeel”, which is the physical sensations, separate from the flavor itself, that one experiences when food is in the mouth. The improvement of the mouthfeel is at least in part because sugar acts as a bulking agent, (i.e., it adds bulk to food), thereby increasing the texture quality. This bulking characteristic also assists in maintaining the volume of a food item.

Additionally, sugar acts as a preservative against food decay and prolongs the shelf life of food products. The preservative quality of sugar works because of its humectant character, which maintains and stabilizes the water content in a food. By stabilizing the water content, sugar slows down the growth of various microbial life forms, such as bacteria, mold, and yeast.

Other characteristics of sugar that may be utilized in the present system and method include its viscosity adding attribute, which is based on its character as a bulking agent, and its anticoagulant attribute, which delays the coagulation of proteins. Coagulation occurs when cream or milk is boiled. One way in which the present system and method prevent coagulation is by preventing boiling, and another is the inclusion of sugar.

Sugar is a broad category of sweet carbohydrates that includes simple sugars or monosaccharides, such as glucose, fructose, and galactose, and so-called “double sugars” or disaccharides, such as sucrose, lactose, and maltose. Double sugars are compounds of two monosaccharides, with sucrose formed from glucose and fructose, lactose formed from glucose and galactose, and maltose formed from two molecules of glucose. There are also saccharide compounds featuring more than two monosaccharides, which are therefore called polysaccharides; however, polysaccharides are generally not referred to as sugars. Sucrose, which is the primary sugar in so-called “table sugar”, is generally extracted from certain plants, such as beets and sugarcane, in the form of raw sugar crystals, whereafter it is refined by washing the sugar crystals and dissolving them into a syrup. This syrup is filtered to improve its purity. The syrup is then desaturated of color by being passed over carbon. The syrup is boiled to increase its concentration and reduce it to crystals. These odorless, colorless, and sweet crystals constitute the table sugar used in the system and method herein, and will be referred to hereafter as simply “sugar”.

Half and half is generally about 4% lactose, which as described previously, is also a sugar. However, the saturation point, which is the concentration level at which the solubility (of half and half) is at equilibrium, is significantly above 4%, and therefore additional sugar can be dissolved.

Half and half may be combined with additional sugar via a blending process. If sugar is merely poured into half and half at room temperature, it will simply settle on the bottom of the receiving vessel, possibly occasioning some passive dissolving. To speed the blending process, energy should be adding into mixture. Energy may be generated in the form of turbulence and/or heat. Thus, the sugar may be added to the half and half in a blender, food processor, or mixing bowl and then the half and half and the sugar may be actively and mechanically blended together. It is important that the heat is applied slowly, to prevent boiling or a Maillard reaction, which is a chemical reaction when protein reacts with sugars, effectively changing the butterfat's flavor and color. Alternatively, the sugar may be mixed with the half and half by using a manually operated utensil, such as a spoon, spatula, or whisk. In addition to mixing and as a prior, concurrent, or subsequent step, the ingredients may be heated in order to facilitate the blending process. The volumetric proportion of half and half to the added sugar is about 9 to 1, respectively. The blending should be sufficiently rigorous to generate enough heat to dissolve the sugar and, if necessary, pasteurize the half & half.

The dissolved blend is then frozen into cubes. Frozen cubes can be formed by pouring liquid in a plastic tray and placing the tray in a freezer, but this is an inefficient process that exposes the frozen cubes to freezer burn and requires the cubes to be forcibly removed from the tray. Another method is to use an icemaker, which separates the cubes from the cube mold using (a small amount of) heat and physically ejects the cubes using ejector blades. The cubes may be 1″ cubed.

The rate at which the blend freezes is important because it affects the size of the (water) crystals. A slower freeze rate results in larger crystals, which are more easily sublimated during the freeze dry process, and a slower freeze rate results in smaller crystals, which can be sublimated without damaging the product texture. The freeze rate, temperature profile, and duration may be conventional for standard ice cream making.

While the blend is freezing, flavors and additional ingredients, such as pistachio, lemon, chocolate, strawberry, etc., may be slowly added so as to ensure that they do not simply sink to the bottom of any given tray.

After the cubes are frozen, they may then be freeze dried. Freeze drying is a process by which material, such as food, is dehydrated at low temperatures. It may include first freezing the food by placing the cubes in a deep freezer, commonly referred to as an IQF (Individual Quick Freeze), lowering the pressure, then removing the (frozen) water content by converting it from a solid to a gas.

Typically, dehydration occurs by increasing the temperature sufficiently to cause evaporation—more precisely, vaporization of liquids from the surface or boiling from the interior. If it is desirable to dehydrate water from a product containing ice, the ice is first melted via heat, and then heated further to bring about the vaporization and boiling described. Unfortunately, the application of heat may have undesirable effects on the structure and composition of the product. However, since the state or phase of matter depends on the pressure as well as the temperature, water may be transitioned from solid to gas with only a minimal use of heat in a process called sublimation.

Sublimation may be effected by reducing the pressure below the substance's “triple point”, which is the point at which the three phases of the substances occur in an equilibrium because the sublimation curve, fusion curve, and vaporization curve intersect. The pressure may be set to the standard in freeze drying. This intersection is important because it provides a pathway by which water can transition between the solid state of ice and the gas state of water vapor without passing through the liquid state, which may damage the product due to the volatility of boiling water, particularly as the boiling water flows through the product medium. As the water in the butterfat-rich product boils, the butterfats may undergo the Maillard reaction, which may have undesirable effects on the manufactured gelato.

Only a small amount of heat is necessary for sublimation, and this heat should be applied slowly in order to damage the product's structure. The temperature and application of heat may be conventional according to standard freeze drying. A slow application of heat allows the energy to be distributed throughout rather than narrowly, intensely, and continuously on any given location. Since the heat will be applied in a low-pressure environment, the heat will mainly occur via conduction or radiation instead of convection.

By reducing the pressure sufficiently, the water molecules have enough room to move away from each other. The minimal amount of heat needed allows the water molecules to overcome their mutual attractive forces, separate, and enter the gas phase. The water vapor, as gas, can be collected and finally separated from the now dehydrated product.

The finished gelato product may be treated with a nitrogen flush and packaged in zip-top laminated stand-up pouches, commonly referred to as flexible packaging. Dessicant powder packs may be included in the packaging to absorb any excess moisture.

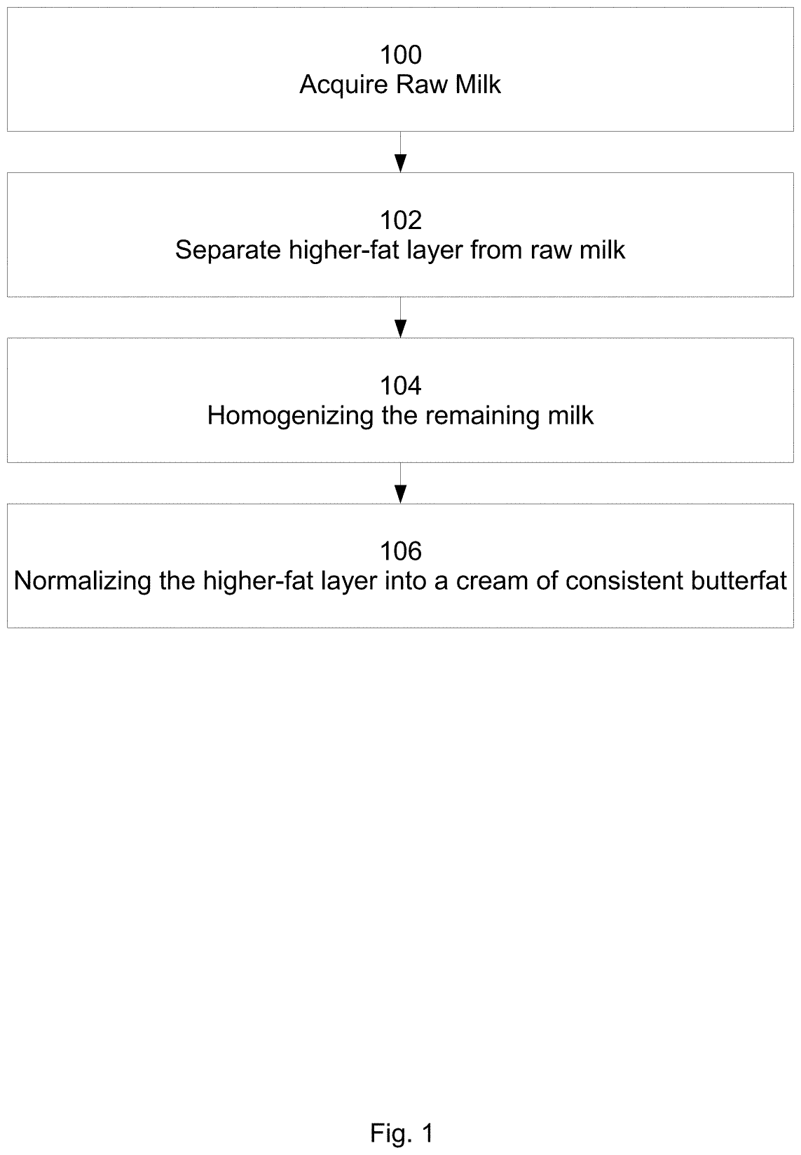

As shown in FIG. 1, the method of manufacturing gelato may include the steps of acquiring raw milk from a milk producing animal 100, separating the higher-fat layer from the raw milk 102, homogenizing the remaining raw milk 104, and normalizing the higher-fat layer into a cream of consistent butterfat content 106.

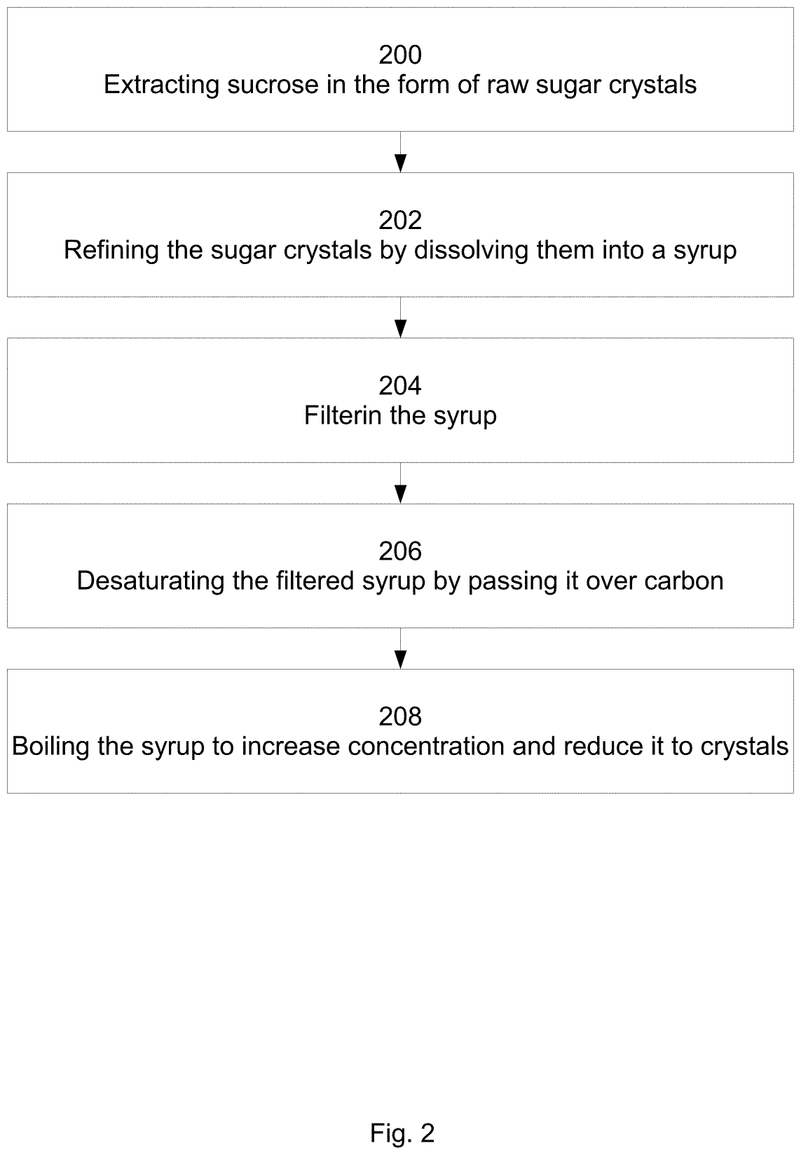

As shown in FIG. 2, the method of manufacturing gelato may include the steps of extracting sucrose from beets or sugarcane in the form of raw sugar crystals 200, refining the sugar crystals by dissolving them into a syrup 202, filtering the syrup 204, desaturating the filtered syrup by passing it over carbon 206, and boiling the filtered and desaturated syrup to increase its concentration and reduce it to crystals 208.

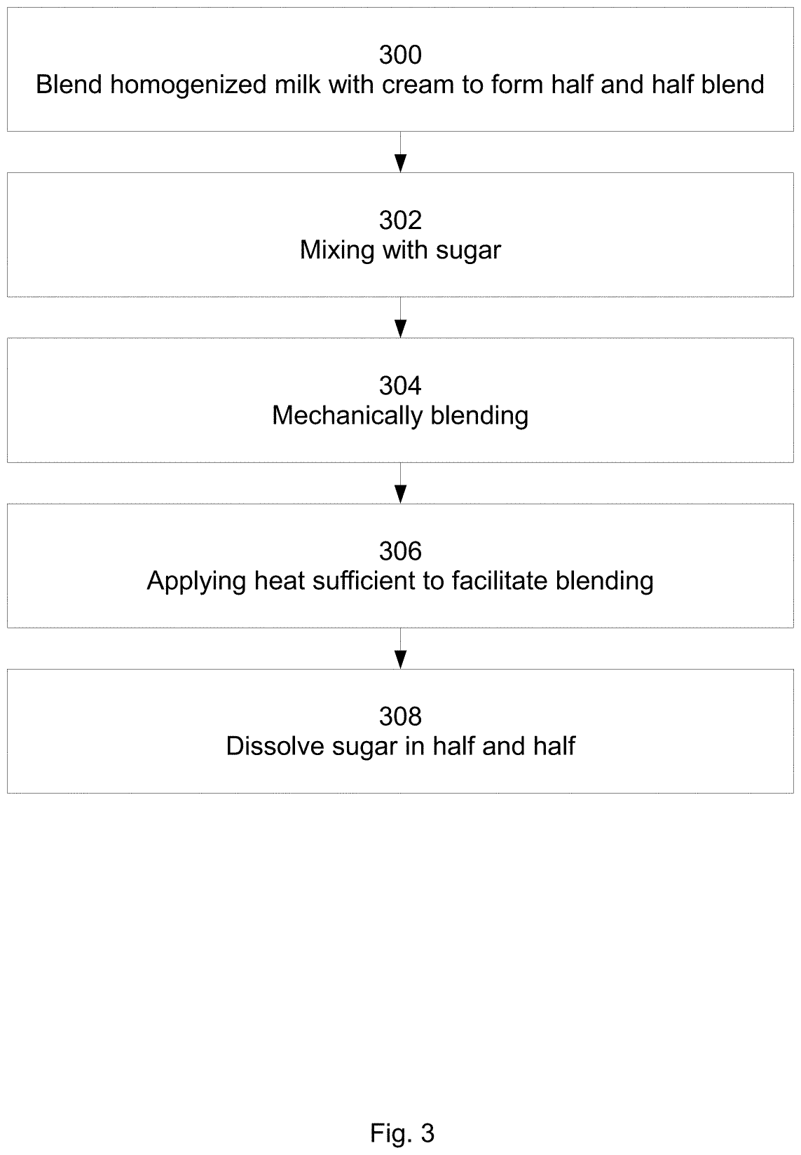

As shown in FIG. 3, the homogenized milk may be blended with the cream to form a half and half blend 300, with the half and half blend having a butterfat content of approximately 20%. The half and half blend may then be further blended with the sugar by adding both to a mixing bowl, food processor, or blender 302, mechanically blending 304, and applying heat sufficient to facilitate blending but insufficient to incur the malliard reaction 306. The blending should continue until the sugar is dissolved in the half and half 308.

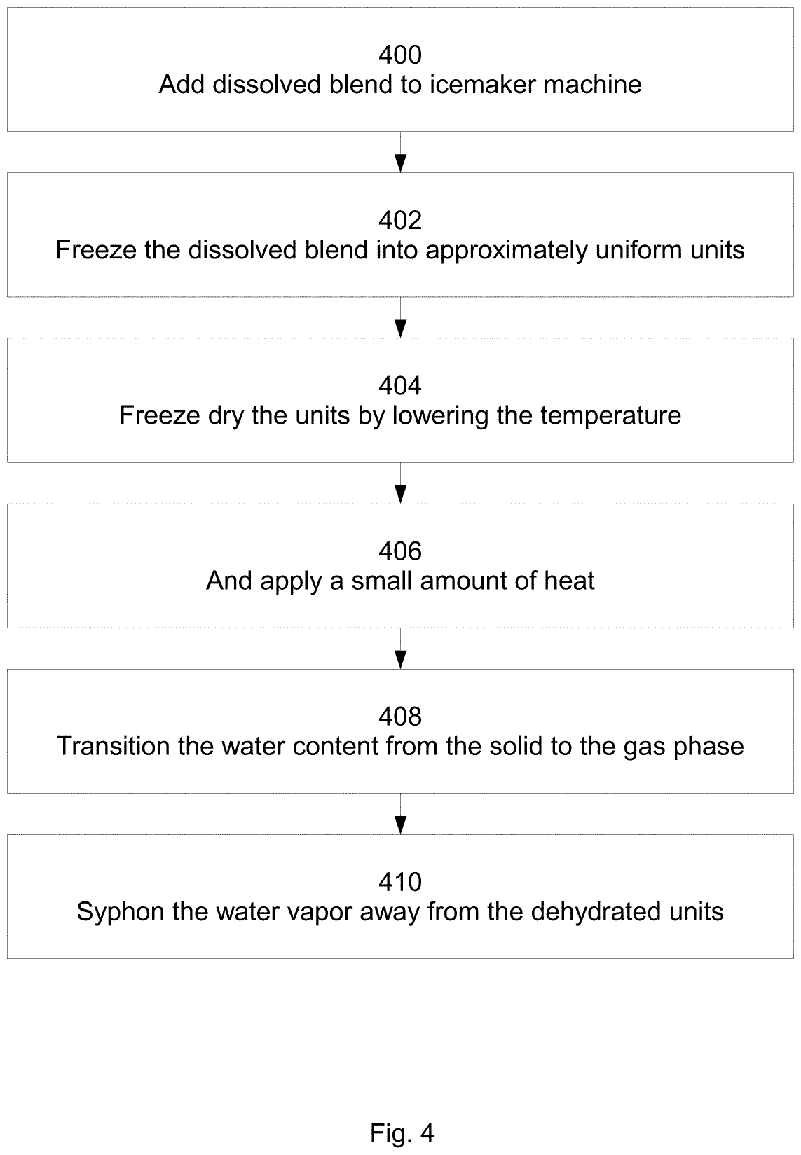

As shown in FIG. 4, the dissolved blend is added to a icemaker machine 400, which freezes the dissolved blend into approximately uniform units 402. The units are then freeze dried by lowering the pressure 404 and applying a small amount of heat 406 to cause the water content in the units to transition between the solid and gas phase 408 without causing the solid to melt into a liquid. The water vapor is then syphoned actively or passively away from the dehydrated units 410.

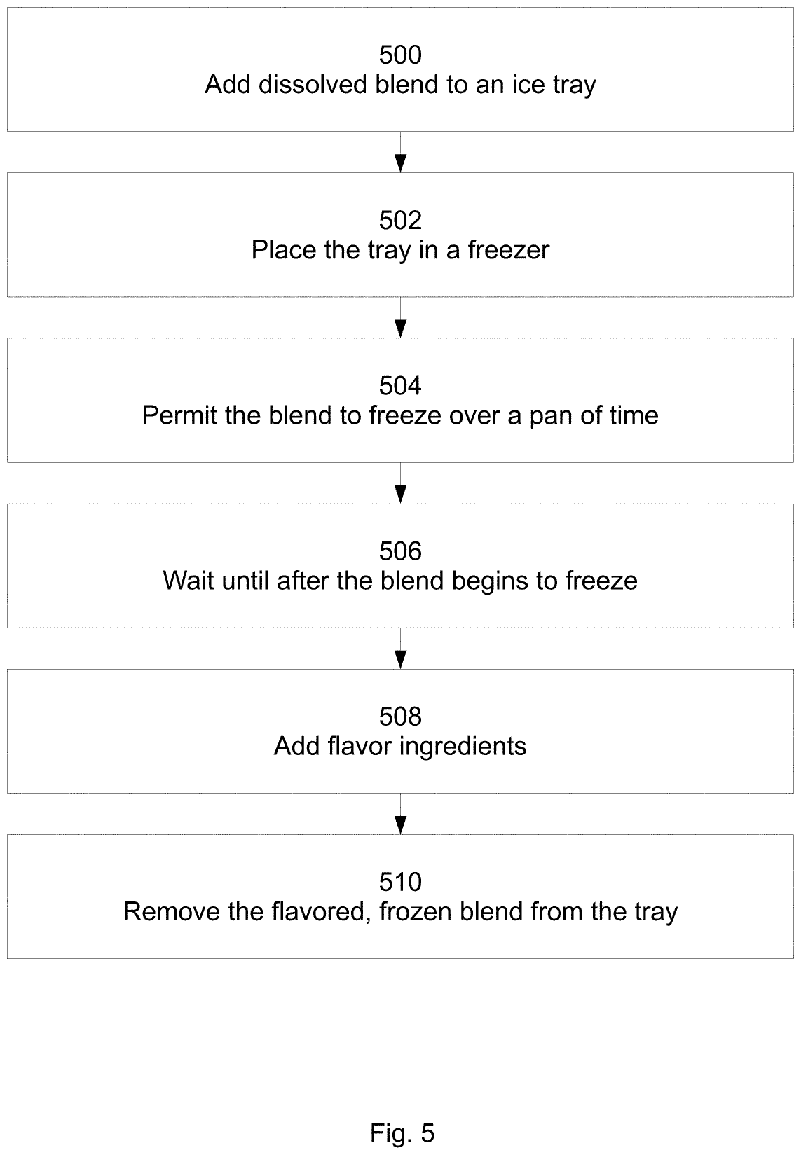

As shown in FIG. 5, the dissolved blend may be added to an ice tray 500, which can be placed in a freezer 502, where it can be permitted to freeze over a span of time 504. Sporadically, or gradually across that span of time, flavor ingredients can be added to the dissolved blend 508. The addition of the flavor ingredients should occur after the blend begins to freeze 506 to prevent the flavor ingredients from simply falling to the bottom. The flavored, frozen blend can then be removed from the ice tray 510.

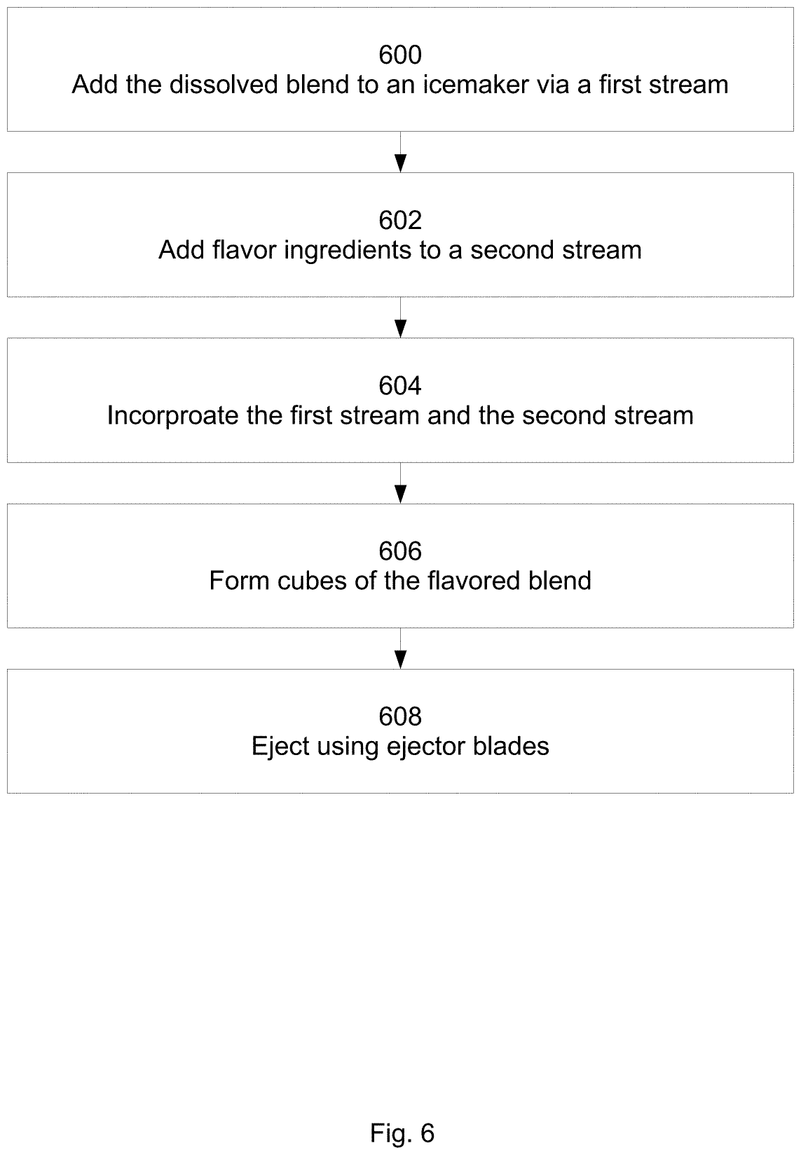

As shown in FIG. 6, the dissolved blend may be added to an icemaker via a first stream 600. Flavor ingredients may be added to a second stream 602. The second stream can be slowly incorporated into the first stream as the first steam is cooled 604. The incorporation can be achieved gradually via conventional flow control mechanisms. The flavored blend will form cubes 606, which may then be physically ejected using ejector blades 608.

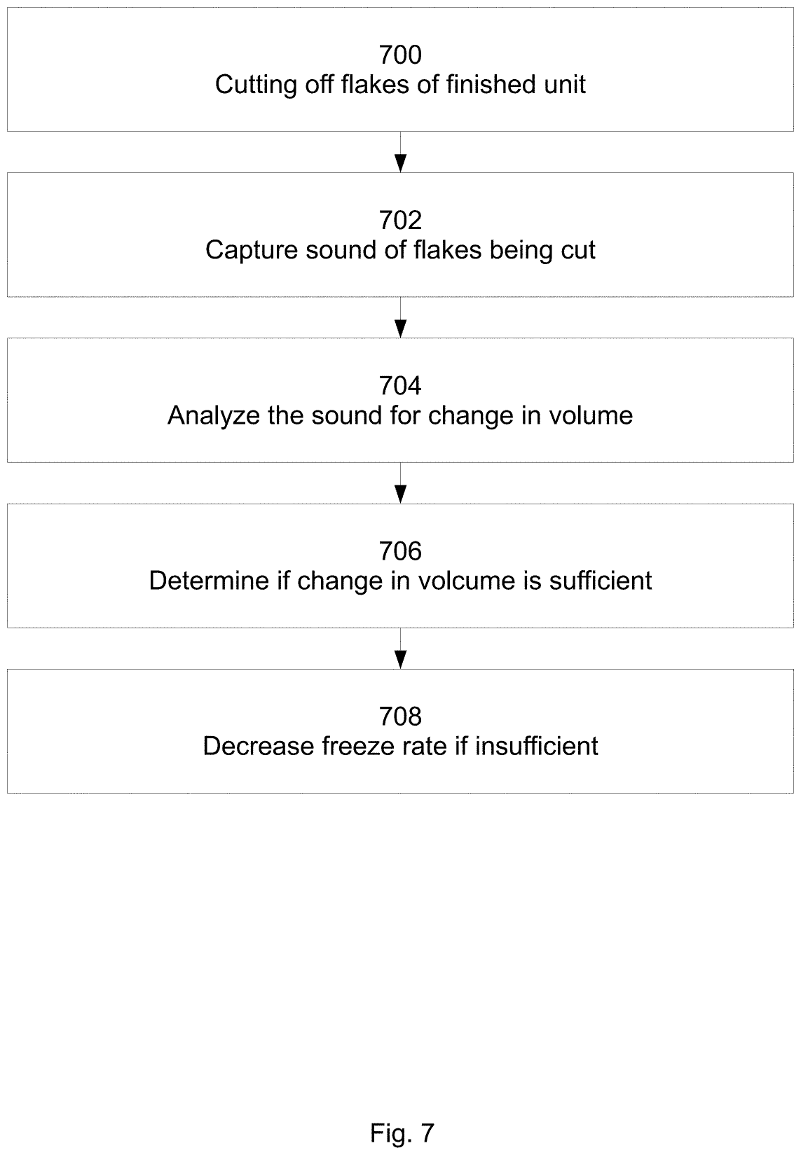

As shown in FIG. 7, the crispiness of the finished units may be measured by cutting flakes off a unit 700, capturing the sound of the flakes being cut 702, and analyzing the sound for the suddenness of the change in volume between pre- and post-cut 704. If the volume change is insufficiently sudden 706, it suggests that the unit is insufficiently crispy, and that therefore the crystals formed during the freeze dry process were too small—therefore, the freeze rate is decreased 708 in order to provide for larger crystals.

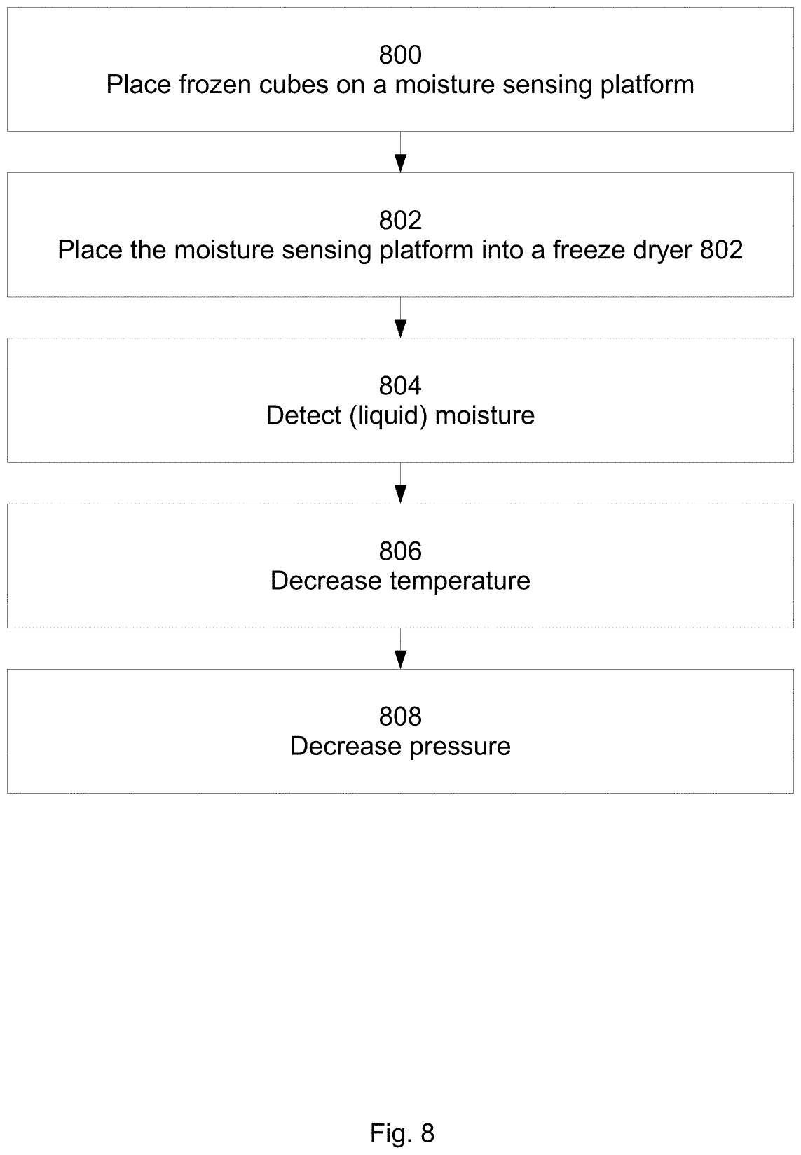

As shown in FIG. 8, the frozen cubes may be placed on top of a moisture sensing platform 800, which may be placed into a freeze dryer 802. The moisture sensing platform is configured to detect liquid moisture. The moisture sensing platform may incorporate a moisture meter, which may operate on the principle of electrical impedence and/or electrical resistance. Additionally or alternatively, moisture may be detected by an infrared camera oriented toward a moisture capture reservoir in the moisture sensing platform, which may simply be a well into which moisture may accumulate if it melts off the frozen cubes. If liquid moisture is detected 804, then the temperature-pressure profile of the freeze dryer is inappropriate for the triple point of the frozen cubes, and so the temperature-pressure is changed by decreasing the temperature 806, or decreasing the pressure 808.

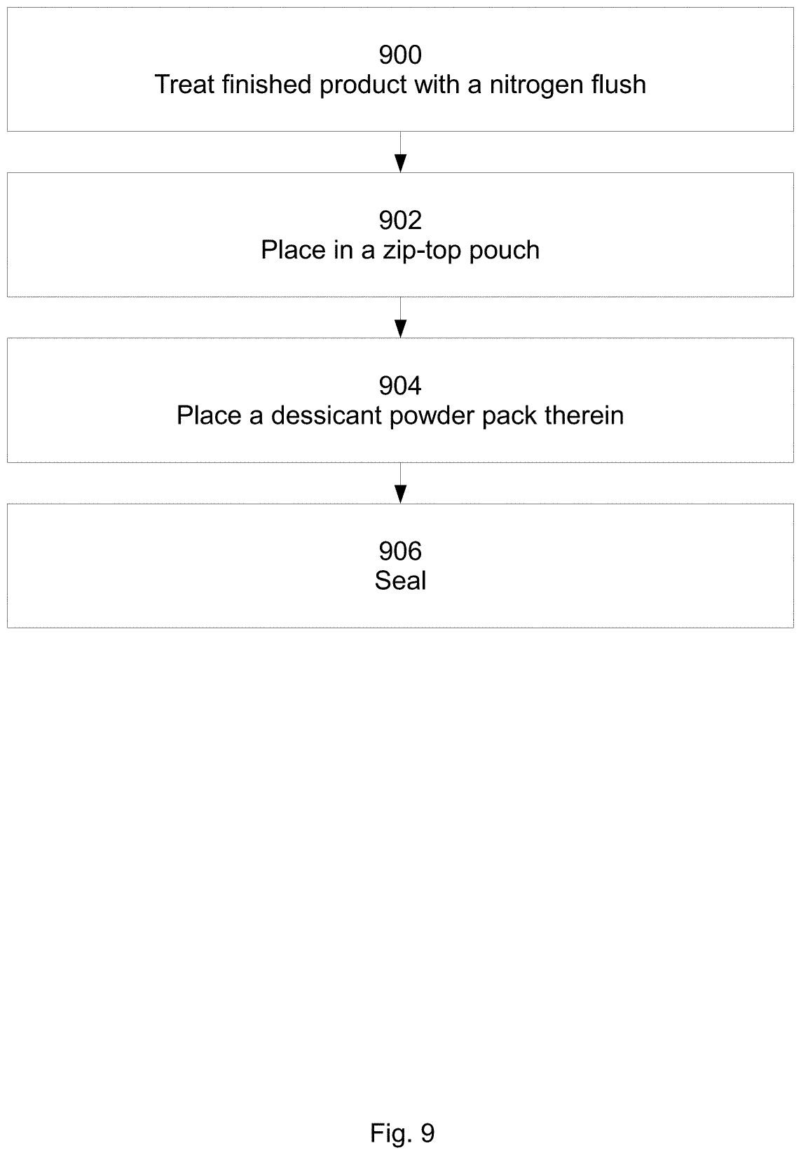

As shown in FIG. 9, the finished product may be treated with a nitrogen flush 900, placed in a zip-top pouch or other sealable, impermeable container 902, into which a dessicant powder pack may be included 904 to absorb any excess moisture, and sealed 906. More than one finished product may be sealed in a given container, and the finished products may be stacked in a row therein. The stacked products may be separated one from another via separators, such as paper or plastic inserts, or protrusions in a food tray, so that the products do not touch each other.

The finished gelato product may be treated with a nitrogen flush and packaged in zip-top laminated stand-up pouches, commonly referred to as flexible packaging. Dessicant powder packs may be included in the packaging to absorb any excess moisture.

A method of manufacturing crispy and shelf-stable gelato may include the steps of gathering cream and sugar, blending the cream and the sugar in a proportion of nine parts cream to one part sugar to produce a blend, freezing the blend into units, each unit being approximately equal in size and shape, and freeze drying the units. The freezing may be completed by an icemaker machine. The units being cubes, spheres, semi-spheres, pyramids, hearts, or crescents. The units may be between one to four inches cubed.

The cream to be blended with the sugar may be between 15-25% butterfat. The cream may more precisely be approximately 20% butterfat. The cream may be made by blending (a) equal proportions of heavy cream and whole milk, (b) approximately ⅔ cup of low-fat milk and ⅓ cup of heavy cream, or (c) approximately 1 cup of whole milk and 1 tablespoon of butter. The sugar blended with the cream may be sucrose. The blending may occur by adding the cream and the sugar into a blender, food processor, or mixing bowl. The blending may involve the application of heat, with the application of heat occurring at an intensity and over a period of time sufficient to prevent a Malliard reaction. The blending may be sufficient to fully dissolve the sugar into the cream.

The method of manufacturing gelato may include the steps of providing cream with a butterfat content between 15-25%, blending the cream with sugar in a volumetric ratio of between 10:1 and 8:1, with the blending being sufficiently rigorous to dissolve the sugar in the cream into a cream-sugar blend but being insufficient to incur a Malliard reaction, adding the cream-sugar blend as a first stream into an icemaker and freezing the cream-sugar blend into approximately uniform frozen units, and transforming water content of the frozen units from a solid phase to a gas phase via freeze-drying the frozen units and syphoning the gas phase water content away, resulting in freeze-dried units. The freeze-dried units may be treated with a nitrogen flush. The freeze-dried units may be packaged in sealable moisture-impermeable bags.

Flavor ingredients may be added as a second stream into the icemaker. The frozen units may be cubes. The frozen units may be stacked together in a row and separated physically via separators.

The crispiness of the freeze-dried units may be ensured by cutting flakes off the freeze-dried units, capturing sound resulting from the cutting of the flakes, determining if a suddenness of a change in volume is sufficient to imply crispiness, and reducing a freeze rate of the icemaker to increase the size of water crystals within the frozen units.

What is claimed is:

1. A method of manufacturing gelato, comprising the steps of:
gathering cream and sugar;
blending the cream and the sugar in a proportion of nine parts cream to one part sugar to produce a blend;
freezing the blend into units, each unit being approximately equal in size and shape; and
freeze drying the units.

2. The method of claim 1, the cream being between 15-25% butterfat.

3. The method of claim 1, the cream being approximately 20% butterfat.

4. The method of claim 1, the cream being made by blending equal proportions of heavy cream and whole milk.

5. The method of claim 1, the cream being made by blending approximately ⅔ cup of low-fat milk and ⅓ cup of heavy cream.

6. The method of claim 1, the cream being made by blending approximately 1 cup of whole milk and 1 tablespoon of butter.

7. The method of claim 1, the sugar being sucrose.

8. The method of claim 1, the blending occurring by adding the cream and the sugar into a blender, food processor, or mixing bowl.

9. The method of claim 8, the blending involving the application of heat, with the application of heat occurring at an intensity and over a period of time sufficient to prevent a Malliard reaction.

10. The method of claim 1, the blending being sufficient to fully dissolve the sugar into the cream.

11. The method of claim 1, the units being cubes, spheres, semi-spheres, pyramids, hearts, or crescents.

12. The method of claim 1, the units being between one to four inches cubed.

13. The method of claim 1, the freezing being completed by an icemaker machine.

14. A method of manufacturing gelato, comprising the steps of:
providing cream with a butterfat content between 15-25%;
blending the cream with sugar in a volumetric ratio of between 10:1 and 8:1, with the blending being sufficiently rigorous to dissolve the sugar in the cream into a cream-sugar blend but being insufficient to incur a Malliard reaction;
adding the cream-sugar blend as a first stream into an icemaker and freezing the cream-sugar blend into approximately uniform frozen units; and
transforming water content of the frozen units from a solid phase to a gas phase via freeze-drying the frozen units and syphoning the gas phase water content away, resulting in freeze-dried units.

15. The method of claim 14, additionally comprising treating the freeze-dried units with a nitrogen flush.

16. The method of claim 14, additionally comprising packaging the freeze-dried units in sealable moisture-impermeable bags.

17. The method of claim 14, additionally comprising adding flavor ingredients as a second stream into the icemaker.

18. The method of claim 14, the frozen units being cubes.

19. The method of claim 18, additionally comprising the steps of stacking the frozen units together in a row and separating the frozen units physically via separators.

20. The method of claim 14, additionally comprising the steps of ensuring the crispiness of the freeze-dried units by cutting flakes off the freeze-dried units, capturing sound resulting from the cutting of the flakes, determining if a suddenness of a change in volume is sufficient to imply crispiness, and reducing a freeze rate of the icemaker to increase the size of water crystals within the frozen units.
